(19) United States
(12) Patent Application Publication
(10) Pub. No.: US 2007/0293422 A1
(43) Pub. Date: Dec. 20, 2007

(54) Beta-Amyloid Inhibitors and Use Thereof

(75) Inventors: Luca Barbero, Chivasso (IT); Pierandrea Esposito, Ivrea (IT); Silvio Traversa, Palazzo Canavese (IT)

Correspondence Address: OBLON, SPIVAK, MCCLELLAND MAIER & NEUSTADT, P.C., 1940 DUKE STREET, ALEXANDRIA, VA, 22314, US

(73) Assignee: APPLIED RESEARCH SYSTEMS ARS HOLDING N.V., Curacao (NL)

(21) Appl. No.: 10/554,372

(22) PCT Filed: Apr. 29, 2004
(86) PCT No.: PCT/EP04/04807

§ 371 Date: Nov. 20, 2006

(30) Foreign Application Priority Data
Apr. 30, 2003 (EP) 03101202.4

(51) Int. Cl. A61K 38/17; A61K 38/08; A61K 38/10; A61P 7/04; C07K 7/06; C07K 7/08; C07K 14/435; A61P 25/28; A61K 38/16

(52) U.S. Cl. 514/12; 514/13; 514/14; 514/15; 530/324; 530/325; 530/326; 530/327; 530/328

(57) ABSTRACT

Peptides and derivatives or analogs thereof are provided for having β-amyloid aggregation inhibitory activity, useful in the treatment and prevention of diseases such as Alzheimer's disease, Dementia pugilistica (including head trauma), Hereditary Cerebral Haemorrhage with amyloidosis of the Dutch type (HCHWA-D) and vascular dementia with amyloid angiopathy.

### FIELD OF INVENTION

The invention relates to the field of amyloid aggregation inhibitor peptides, particularly their use in the treatment of diseases such as Alzheimer's disease, Dementia pugilistica (including head trauma), Hereditary Cerebral Haemorrhage with amyloidosis of the Dutch type (HCHWA-D) and vascular dementia with amyloid angiopathy.

### BACKGROUND OF THE INVENTION

Alzheimer's disease (AD), first described by the Bavarian psychiatrist Alois Alzheimer in 1907, is a progressive neurological disorder that begins with short-term memory loss and is characterized by a progressive decline in cognitive function and behavior. Progression of the disease leads to disorientation, impairment of judgment, reasoning, attention and speech and, ultimately, dementia. The course of the disease usually leads to death in a severely debilitated, immobile state between four and 12 years after onset. AD has been estimated to afflict 5 to 11 percent of the population over age 65 and as much as 47 percent of the population over age 85. The societal cost for managing AD is very high, primarily due to the extensive custodial care required for AD patients. Despite continuous efforts aimed at understanding the physiopathology of AD, there is currently no treatment that significantly retards the progression of the disease.

Pathologically, AD is characterized by the presence of distinctive lesions in the victim's brain, revealed on autopsy. These brain lesions include abnormal intracellular filaments called neurofibrillary tangles (NTFs) and extra cellular deposits of amyloidogenic proteins in senile, or amyloid, plaques. Amyloid deposits are also present in the walls of cerebral blood vessels of AD patients. The major protein constituent of amyloid plaques has been identified as a 4.3 kiloDalton peptide called β-amyloid peptide (Aβ) (Selkoe et al., 1997).

Genetic and neuropathological studies suggest that the processing of amyloid precursor protein (APP) to yield Aβ, and its subsequent aggregation, play important roles in the pathology of Alzheimer's disease. Sequential cleavage of APP α-, followed by β-secretases yields to two major species of Aβ ending at residue 40 (Aβ₁₋₄₀) or 42 (Aβ₁₋₄₂) and these molecules tend to aggregate to form oligomers, AD diffusible ligands (ADDLs) and protofibrils, which have been suggested to cause neuronal dysfunction in the brains of AD patients. These Aβ aggregates may induce neuronal injury directly by acting on synapses, or indirectly by activating microglia and astrocytes (Hardy et al., 2002).

Patients with hereditary cerebral haemorrhage with amyloidosis-Dutch-type (HCHWA-D), which is characterized by diffuse β-amyloid deposits within the cerebral cortex and cerebrovasculature, have been shown to present mutations in the APP gene leading to an amino acid substitution within Aβ (Levy et al., 1990).

Aβ has also been implicated in vascular dementia with amyloid angiopathy (Maury et al., 1995) and dementia pugilistica (Jordan et al., 2000).

The APP gene maps to chromosome 21, thereby providing an explanation for the β-amyloid deposition seen at an early age in individuals with Down's syndrome, which is caused by trisomy of chromosome 21 (Mann et al., 1988).

Considerable evidence has accumulated that the pathogenicity of Aβ results from a change in protein conformation (Soto et al., 1999). It is believed that a critical event leading to pathology in Alzheimer's disease, Vascular dementia with amyloid angiopathy and HCHWA-D is the refolding of a natural and non-pathogenic protein, to yield a pathogenic form. The refolding alters the secondary and tertiary structure of the protein without changing its primary structure. The process that lead to amyloid aggregation is poorly understood and only some step forward have been made in the mechanism elucidation (Harper et al., 1997).

Amyloid is a generic term that is applied to fibrillar aggregates that have a common structural motif: a β-pleated sheet conformation. These aggregates exhibit special tinctorial properties, including the ability to emit a green birefringent glow after staining with Congo red, and the capacity to bind the fluorochrome thioflavin (Soto et al., 1995). These tinctorial properties form the basis of assays used to detect β-amyloid deposits.

Several different treatment strategies have been developed to target sequential events originating from Aβ synthesis (Xia et al., 2003).

One approach to the treatment and prevention of Alzheimer's disease has been to develop agents for blocking Aβ aggregation for preventing Aβ aggregate-mediated downstream deleterious events.

Amongst other such agents, short peptides having some sequence homology to the natural protein sequence believed to be involved in amyloid formation, but also having one or more amino acids that disfavor or destabilize the formation of β-pleated sheet conformations have been developed (WO 96/39834, WO 01/34631). Others have developed short peptides having some sequence homology to the natural protein sequence believed to be involved in amyloid formation and carrying at one end, either bulky chemical modifying groups (U.S. Pat. No. 6,319,498) or stretches of charged amino acids (KKKK or EEEE) (Lowe et al., 2001).

These results further support the concept of preventing Aβ aggregation as a potential therapeutic tool for Alzheimer's disease and other amyloid diseases. However, the desired site of action for treatment of many amyloid-related disorders is in the brain, and peptides, like many other molecules, may have difficulty penetrating the blood brain barrier (BBB). It has also been proposed inhibitory peptides for preventing the formation of extended beta-sheets that are composed of a beta-strand forming region, followed or preceded by a distinct membrane-penetrating section (WO 01/07473).

Penetratin is a 16-mer peptide (pAntp) derived from the third helix domain of Antennapedia homeoprotein (amino acids from 43 to 58) and known as a cell translocation sequence (Derossi et al., 1994). Due to these translocation properties, this sequence is currently used as membrane translocation vector to shuttle hydrophilic molecules (WO 00/29427), proteins, peptides (WO 01/09170; WO 00/63246), oligopeptides, antibodies (FR 2829240) and oligonucleotides (WO 98/38861; WO 02/062989) into live cells in vitro and in vivo.

Furthermore, pAntp and its derivatives have shown to be able to cross some physiological barriers, such as the Blood Brain Barrier (Rousselle et al., 2000).

Therefore, the development of new beta-amyloid inhibitory agents, including peptides that are able to cross the BBB, would have several therapeutic advantages.

### SUMMARY OF THE INVENTION

It is an object of the invention to provide β-amyloid inhibiting substances which are suitable for the treatment of and/or prevention of and/or delaying the progression of beta-amyloid related disorders, notably, Alzheimer's Disease.

It is also an object of the invention to provide substances which are suitable for reducing or inhibiting beta-amyloid aggregation.

In a first aspect, the invention provides a peptide of formula I (SEQ ID NO: 1):
X₁[Lys X₂ X₃ Phe Gln]ₘArg Gln Ile[Lys X₄ Pro Phe Gln]ₙX in which
X₁ is absent or is an acetyl group;
X₂ and X₄ are independently selected from Isoleucine or Leucine;
X₃ is selected from Proline and Tryptophane;
X is a peptidic moiety of a length selected from 1, 2, 3, 4, 5, 6, 7 and 8 amino acids containing at least one basic amino acid and which is amidated at the C-terminus;
m is an integer selected from 0 and 1;
n is an integer selected from 1 and 2;
   as well as salt and any derivative, analogue or conjugate thereof.

In a second aspect, the invention provides a peptide according to Formula I for use as a medicament.

In a third aspect, the invention provides a pharmaceutical composition comprising a compound of Formula I, together with a pharmaceutically acceptable excipient or carrier.

In a fourth aspect, the invention provides a use of a compound of Formula II (SEQ ID NO: 3):
X₁[Lys X₂ X₃ Phe Gln]ₘArg Gln Ile[Lys X₄ X₅ Phe Gln]ₙX in which
X₁ is absent or is an acetyl group;
X₂ and X₄ are independently selected from Isoleucine and Leucine;
X₃ and X₅ are independently selected from is Proline and Tryptophane;
X is a peptidic moiety of a length selected from 1, 2, 3, 4, 5, 6, 7 and 8 amino acids containing at least one basic amino acid and which is amidated at the C-terminus;
m is an integer selected from 0 and 1;
n is an integer selected from 1 and 2;
   as well as derivatives thereof and mixtures of these, as well as salts thereof for the preparation of a medicament for the treatment or prevention of a disease or condition selected from Alzheimer's disease, Dementia pugilistica (including head trauma), Hereditary Cerebral Haemorrhage with amyloidosis of the Dutch type (HCHWA-D) and vascular dementia with amyloid angiopathy.

In a fifth aspect, the invention provides a use of a compound of Formula (II) for the preparation of a medicament for the treatment or prevention of a disease associated with abnormal protein folding into amyloid and amyloid-like deposits.

In a sixth aspect, the invention provides a method of treating a disease associated with abnormal protein folding into amyloid and amyloid-like deposits, including Alzheimer's disease, Dementia pugilistica (including head trauma), Hereditary Cerebral Haemorrhage with amyloidosis of the Dutch type (HCHWA-D) and vascular dementia with amyloid angiopathy, comprising administering to a patient in need thereof an effective amount of a compound of Formula (II).

### DETAILED DESCRIPTION OF THE INVENTION

The following paragraphs provide definitions of various chemical moieties and terms, and are intended to apply uniformly throughout the specification and claims unless an otherwise expressly set out definition provides a different definition.

The term “peptide” is ordinarily applied to a polypeptidic chain containing from 3 to 30 or more contiguous amino acids, usually from 3 to 20 contiguous amino acids. Such peptides can be generated by methods known to those skilled in the art, including partial proteolytic cleavage of a larger protein, chemical synthesis, or genetic engineering.

The expression “derivative or analogue” means any compound the chemical structure of which contains modifications with respect to the parent peptide, but which maintains at least 50%, more preferably at least 75%, most preferably at least 90% of the biological activity of a compound of Formulae I or II.

The term “derivatives” as herein used refers to derivatives which can be prepared from the functional groups present on the lateral chains of the amino acid moieties or on the N-/ or C-terminal groups according to known methods. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups. The term “derivatives” includes also “chiral derivatives”.

The term “fragment” as herein used refers to shorter derivatives of amyloid inhibitors of the invention which maintain at least 50%, more preferably at least 75%, most preferably at least 90% of the biological activity of a compound of Formulae I or II.

The term “conjugates” as herein used refers to a peptide wherein a beta amyloid inhibitor of the invention is linked (e.g. covalently) to either another beta-amyloid inhibitor or to a fragment thereof. The linkage between the two or more beta amyloid inhibitor sub-units can be direct or indirect, via a linker moiety. Direct linkage may occur through any convenient functional group on the peptide of the invention such as hydroxy, carboxy, amino group, preferably at one terminus. The direct linkage can be performed, for example, during the solid synthesis, the resulting conjugate being one continuous peptide. Indirect linkage can occur through a linking group. Examples of linking group include multifunctional alkyl, aryl, aralkyl, organic polymers or short peptidic moieties of 1 to 4 residues.

Examples of “conjugates” include peptides wherein a peptide of the invention is linked together with at least one copy of a peptide of the invention or a fragment thereof, and also peptides wherein a peptide of the invention is linked to another known beta-amyloid inhibitor (β-AI) in order to improve properties of the known beta-amyloid inhibitor (e.g. improved inhibitory activity on beta-amyloid aggregation, improved pharmacokinetic properties, reduced toxicity etc). One preferred example of conjugate is a conjugate formed by the covalent linkage of a beta-amyloid inhibitor (β-AI) to the C-terminus of a peptide of the invention. Examples of known beta-amyloid inhibitors (β-AIs) are available to the person skilled in the art and can be found, for example, in Talaga, 2001. One example of a class of β-AIs is represented by beta-sheet breakers (BSBs), including BSB1, i.e. SEQ ID NO: 5 (WO 01/34631). One example of “conjugate” of the invention is a peptide of SEQ ID NO: 6.

The term “salts” herein refers to both salts of carboxyl groups and to acid addition salts of amino groups of the peptides, polypeptides, or analogs thereof, of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “chiral derivative” refers to any substitution of a normal amino acid (L-enantiomer) by the corresponding D-enantiomer.

The term “peptidic moiety” refers to a peptidic sequence of at least one amino acid that is bound via a peptidic bond. The length of the peptidic moiety is expressed by the number of amino acids present in the peptidic sequence. Examples of peptidic moieties are peptidic sequences of 1 to 8 amino acids, preferably more than 3 amino acids, most preferably from 5 to 8 amino acids.

The term “basic amino acids” refers to amino acids positively charged. Examples of basic amino acids are Lysine (Lys), Arginine (Arg), Histidine (His) and derivatives thereof. Examples of “peptidic moiety” “containing at least one basic amino acid” are peptidic moieties that have one or more basic residues such as Lysine, Arginine, Histidine or derivatives thereof, within its sequence.

When more than one basic residue are present, they can be at consecutive positions or at alternating positions within the sequence of the peptidic moiety. When the basic amino acids are at alternating positions, one or more non-basic amino acid, preferably neutral such as Asparagine (Asn), Methionine (Met) or Tryptophane (Trp) can be intercalated between the basic amino acids.

The following three letter code or one letter code are employed for the following amino acids:

Arginine (Arg, R), Asparagine (Asn, N), Glutamine (Gln, Q), Histidine (His, H), Isoleucine (Ile, I), Leucine (Leu, L), Methionine (Met, M), Phenylalanine (Phe, F), Proline (Pro, P) and Tryptophane (Trp, W).

The term “acetyl” (Ac) defines the group —CH(O)OH. Acetylated peptides at the N-terminus are peptides which have an “acetyl” group on the nitrogen atom of the first amino acid.

“Fibrils” or “amyloid fibrils” refer to fibrillar aggregates that form the amyloid plaques. These “fibrils” can be characterized by several of their properties such as birefringence in polarized microscopy, a property that increased intensely after staining with Congo red dye, Thioflavine T fluorescence increase or extensive beta-sheet structure as revealed by far-UV CD and IR spectroscopy.

The term “β-amyloid inhibiting substances” refers to substances that are able to reduce, block or prevent the formation and/or extension of amyloid fibrils. This term also includes substances that are able to dissolve, even partially, already formed fibrils.

The term “β-amyloid like deposits” refers to fibrillar deposits or fibrils that have the same aspect as amyloid fibrils by electron micrograph of negative-stained samples but are formed by a fragment of a non-amyloid related peptide that is a potentially amylogenic sequence motif, i.e. a fragment of peptide that has not been classified as “amyloidogenic peptide”.

Peptide of the invention can be mimetics (also called peptidomimetics) of SEQ ID NO: 4, 6, 7, 8 or 9 in which the nature of peptide has been chemically modified at the level of amino acid side chains, of amino acid chirality, and/or of the peptide backbone. These alterations are intended to provide beta amyloid inhibiting agents having similar or improved therapeutic, diagnostic and/or pharmacokinetic properties.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are known in the art (WO 02/10195; Villain et al., 2001).

The techniques for the synthesis and the development of peptide mimetics, as well as non-peptide mimetics, are well known in the art (Golebiowski et al., 2001; Kim et al., 2000). Various methodology for incorporating unnatural amino acids into proteins, using both in vitro and in vivo translation systems, to probe and/or improve protein structure and function are also disclosed in the literature (Dougherty, 2000).

The peptides of the present invention can be in other alternative forms which can be preferred according to the desired method of use and/or production, for example as active fragments, salts, derivatives or conjugates.

The compounds of the invention may be prepared by any well-known procedure in the art, including chemical synthesis technologies.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzy oxycarbonyl), Bzl (benzyl), Fmoc (9-fluorenylmethoxycarbonyl), Mbh (4,4′-dimethoxy dibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The compounds of the invention are β-amyloid inhibitor peptides.

β-amyloid inhibiting activity can be detected using, for example, an in vitro assay, such as that described by (Levine et al., 1993) which measures the ability of test compounds to prevent amyloid fibril formation. Results are reported in the Examples.

Amyloid fibrils are cytotoxic, inducing cell death by apoptosis (Yankner, 1996). Compounds of the invention can be tested for their ability to prevent cell death induced by amyloid fibrils.

In a preferred group of peptides of Formula I, X is a peptidic moiety of a length selected from 5, 6, 7 and 8 amino acids containing at least one basic amino acid such as Lysine or Arginine. One example of a preferred X is a peptidic moiety of SEQ ID NO: 2:
Asn X₅ X₆ Met X₇ Trp X₈ X₉—NH₂
wherein X₅, X₆, X₇, X₈ and X₉ are independently selected from Arginine and Lysine; or a derivative or analog thereof. Another example of a preferred peptidic moiety is of SEQ ID NO: 10.

In another preferred group of peptides of Formula I, m is 0 and n is 1.

In another preferred group of peptides of Formula I, X₁ is acetyl.

In another preferred group of peptides of Formula I, m is 0 and n is 2.

In another preferred group of peptides of Formula I, m is 1 and n is 1.

In another preferred group of the invention, the peptides of Formula I are selected from SEQ ID: 7 and SEQ ID: 8.

Compounds of Formula I may be used for the treatment of a disease.

In a further embodiment of the invention, is provided a pharmaceutical composition comprising a peptide of Formula I and a pharmaceutically acceptable excipient, diluent or carrier.

Another embodiment of the invention provides the use of a compound of Formula II (SEQ ID NO: 3) described above as well as derivatives, analogies or conjugates thereof and mixtures of these, as well as salts thereof for the preparation of a medicament for the manufacture of a medicament for the treatment or prevention of a disease or condition selected from Alzheimer's disease, Dementia pugilistica (including head trauma), Hereditary Cerebral Haemorrhage with amyloidosis of the Dutch type (HCHWA-D) and vascular dementia with amyloid angiopathy.

In a preferred group of peptides according to Formula II, X₅ is Tryptophane.

In another preferred group of peptides according to Formula II, peptides are according to SEQ ID NO: 1.

In another preferred group of peptides according to Formula II, X₄ is Isoleucine.

In another preferred group of peptides according to Formula II, m is 0 and n is 1.

In another preferred group of peptides according to Formula II, X is a peptidic moiety of a length selected from 5, 6, 7 and 8 amino acids containing at least one basic amino acid such as Lysine or Arginine. One example of a preferred X is a peptidic moiety of SEQ ID NO: 2:
Asn X₅ X₆ Met X₇ Trp X₈ X₉—NH₂
wherein X₅, X₆, X₇, X₈ and X₉ are independently selected from Arginine and Lysine; or a derivative or analog thereof. A example of a particularly preferred peptidic moiety is of SEQ ID NO: 10.

In another preferred group of peptides according to Formula II, X₅ is Tryptophane, X is the peptidic moiety of SEQ ID NO: 2 as defined above, m is 0 and n is 1.

In another preferred group of the invention, the peptides of Formula II are selected from the following group:

SEQ ID NO: 7, SEQ ID NO: 8 and SEQ ID NO: 9.

In another preferred group of the invention, the peptide of Formula II is of SEQ ID NO: 4.

Specifically, the compounds of Formulae I or II are suitable for use in the preparation of a medicament for the treatment or prevention of beta-amyloid related disorders, such as beta-amyloid aggregation-related disorders, including Alzheimer's disease, Dementia pugilistica (including head trauma), Hereditary Cerebral Haemorrhage with amyloidosis of the Dutch type (HCHWA-D) and vascular dementia with amyloid angiopathy.

Still another embodiment of the present invention, is a method for treating or preventing neurodegenerative disorders such as Alzheimer's disease, Dementia pugilistica (including head trauma), Hereditary Cerebral Haemorrhage with amyloidosis of the Dutch type (HCHWA-D) and vascular dementia with amyloid angiopathy.

A further embodiment of the invention is a method for treating or preventing beta-amyloid disorders wherein the method comprises administering an effective dose of the above-mentioned peptides and derivatives thereof to a subject in the need thereof, wherein the subject can be human or animal, preferably human.

Still a further embodiment of the invention comprises the administration of at least a compound of the invention in a regimen coordinated with at least another beta-amyloid inhibitor, for simultaneous, sequential or separate use.

In another embodiment of the invention, a compound of the invention is fused to a carrier molecule, a peptide or a protein that promotes the crossing of the blood brain barrier (“BBB”). This serves for proper targeting of the molecule to the site of action in those cases, in which the CNS is involved in the disease. Modalities for drug delivery through the BBB entail disruption of the BBB, either by osmotic means or biochemically by the use of vasoactive substances such as bradykinin. Other strategies to go through the BBB may entail the use of passive diffusion and the use of endogenous transport systems, including carrier-mediated transporters such as glucose and amino acid carriers; receptor-mediated transcytosis for insulin or transferrin; adsorptive-mediated transcytosis. Strategies for drug delivery behind the BBB further include intra-cerebral implantation.

The compounds of the invention prevent the aggregation of Aβ associated with the onset and progression of Alzheimer's disease, Dementia pugilistica (including head trauma), Hereditary Cerebral Haemorrhage with amyloidosis of the Dutch type (HCHWA-D) and vascular dementia with amyloid angiopathy. In a preferred method of use of the compounds, administration of the compounds is by injection or infusion, at periodic intervals. The administration of a compound of the invention should preferably begin before any symptoms are detected in the patient and should continue thereafter. Patients at a high risk for developing Alzheimer's disease, Hereditary Cerebral Haemorrhage with amyloidosis of the Dutch type (HCHWA-D) and vascular dementia with amyloid angiopathy include those with a familial history of these diseases.

In a further embodiment, compounds according to Formulae I or II are suitable for the treatment or prevention of beta-amyloid related disorders, such as beta-amyloid aggregation-related disorders, including Alzheimer's disease, Dementia pugilistica (including head trauma), Hereditary Cerebral Haemorrhage with amyloidosis of the Dutch type (HCHWA-D) and vascular dementia with amyloid angiopathy.

The compounds of the invention may be isolated and purified as salts. Such salts fall within the scope of the invention. For the purposes of administration to a patient, it is desirable that the salts be pharmaceutically acceptable.

The compounds of the invention can be administered as salts. Such salts include: salts of carboxyl groups or acid addition salts of amino groups of the peptide of the invention. Salts of a carboxyl group may be formed by means known in the art and include inorganic salts, for example, sodium, calcium, ammonium, ferric or zinc salts, and the like, and salts with organic bases as those formed, for example, with amines, such as tri-ethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid.

Pharmaceutical compositions comprising at least one peptide of the invention include all compositions wherein the peptide(s) are contained in an amount effective to achieve the intended purpose. In addition, the pharmaceutical compositions may contain suitable pharmaceutically acceptable carriers comprising excipients and auxiliaries which facilitate processing of the active compounds into preparations which can be used pharmaceutically. Suitable pharmaceutically acceptable vehicles are well known in the art and are described for example in Gennaro et al, 2000, a standard reference text in this field. Pharmaceutically acceptable vehicles can be routinely selected in accordance with the mode of administration and the solubility and stability of the peptides. For example, formulations for intravenous administration may include sterile aqueous solutions which may also contain buffers, diluents and other suitable additives. The use of biomaterials and other polymers for drug delivery, as well the different techniques and models to validate a specific mode of administration, are disclosed in literature (Luo et al., 2001; Cleland et al., 2001).

The above-mentioned peptides and derivatives of the present invention may be administered by any means that achieves the intended purpose. For example, administration may be by a number of different routes including, but not limited to subcutaneous, intravenous, intradermal, intramuscular, intraperitoneal, intra-cerebral, intrathecal, intranasal, oral, rectal, transdermal, intranasal or buccal. Preferably the compounds of the invention are administered by subcutaneous, intramuscular or intravenous injection or infusion.

Parenteral administration can be by bolus injection or by gradual perfusion over time. A typical regimen for preventing, suppressing, or treating amylin misfolding related disorders, comprises either (1) administration of an effective amount in one or two doses of a high concentration of inhibitory peptides in the range of 0.5 to 10 mg of peptide, more preferably 0.5 to 5 mg of peptide, or (2) administration of an effective amount of the peptide in multiple doses of lower concentrations of inhibitor peptides in the range of 10-1000 μg, more preferably 50-500 μg over a period of time up to and including several months to several years. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Preparations for parenteral administration include sterile aqueous or non-aqueous solutions, suspensions, and emulsions, which may contain auxiliary agents or excipients which are known in the art. Suitable formulations for parenteral administration include aqueous solutions of the active compounds in water-soluble form, for example, water-soluble salts. In addition, suspension of the active compound as appropriate oily injections suspensions may be administered.

Depending on the intended route of delivery, the compounds may be formulated as injectable or oral compositions. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a pre-determined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavouring agent such as peppermint, methyl salicylate, or orange flavouring.

Injectable compositions are typically based upon injectable sterile saline or phosphate-buffered saline or other injectable carriers known in the art.

The above-described components for orally administered or injectable compositions are merely representative. Further materials as well as processing techniques and the like are known to the skilled practitioner (Gennaro et al., 2000).

The compounds of this invention can also be administered in sustained release forms or from sustained release drug delivery systems. A description of representative sustained release materials is also known to the skilled practitioner (Karsa et al., 1993; Yacobi et al., 1998).

By “effective amount”, is meant an mount sufficient to achieve a concentration of peptide(s) which is capable of slowing down or inhibiting the formation of amylin deposits, or of dissolving preformed deposits. Such concentrations can be routinely determined by those of skill in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like. It will also be appreciated by those of skill in the art that the dosage may be dependent on the stability of the administered peptide. A less stable peptide may require administration in multiple doses.

The expression “Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution.

Besides the pharmaceutically acceptable carrier, the compositions of the invention can also comprise minor amounts of additives, such as stabilizers, excipients, buffers and preservatives.

It will be appreciated that where typical or preferred experimental conditions for preparing compounds of Formulae I or II (i.e., reaction temperatures, time, moles of reagents, solvents, etc.) are given, other experimental conditions can also be used unless otherwise stated. Optimum reaction conditions may vary with the particular reactants or solvent used, but such conditions can be determined by one skilled in the art by routine optimisation procedures.

The compounds of the invention may be prepared using methods of peptide synthesis known to the skilled practitioner (Bodanzski, 1993; Weng et al., 2000).

In a preferred embodiment, the compounds of the invention are synthesized using solid-phase methods.

The present invention has been described with reference to the specific embodiments, but the content of the description comprises all modifications and substitutions, which can be brought by a person skilled in the art without extending beyond the meaning and purpose of the claims.

The invention will now be described by means of the following Examples, which should not be construed as in any way limiting the present invention. The Examples will refer to the Figures specified here below.

### BRIEF DESCRIPTION OF THE DRAWINGS

FIG. 1 shows the effect of peptides of the invention on amyloid Aβ₁₋₄₂ aggregate formation (SEQ ID NO: 11).

The percentage of formed fibrils after 2 days incubation with 110 μM of Aβ₁₋₄₂ (SEQ ID NO: 11) at 37° C. is represented versus the concentration of the peptides of the invention (in μM). 100% of formed fibrils correspond to the fibrils formed in presence of Aβ₁₋₄₂ alone. Triangles represent data for pAntp (SEQ ID NO: 4) and squares represent data for pAntp-BSB1 (SEQ ID NO: 6). The percentage of formed fibrils for peptides of the invention is compared to that obtained for a known beta-sheet breaker, BSB1 of SEQ ID NO: 5 (Lozenges). Data are the result of three independent experiments in duplicate.

### ABBREVIATIONS

The following abbreviations are hereinafter used in the accompanying examples:

DMSO (dimethyl sulfoxide), min (minute), hr (hour), g (gram), mM (millimolar), ml (milliliter), nm (nanometer), μg (micrograms), μl (micro liters), μM (micro molar), rt (room temperature).

### EXAMPLES

The invention will be illustrated by means of the following examples which are not to be construed as limiting the scope of the invention.

The following examples illustrate preferred compounds according to Formulae I or II, and methods for determining their biological activities.

Synthetic pAntp (1-16) (SEQ ID NO: 4), BSB1 (SEQ ID NO: 5) and peptide of SEQ ID NO: 6 were synthesized in solid phase. Ab₁₋₄₂ (SEQ ID NO: 11), MW 4513 Da was purchased from BACHEM (H-1368.1000).

### Example 1

### Synthesis of Compounds of the Invention

Peptides of the invention are synthesized in solid phase by Fmoc chemistry. Peptides were purified by HPLC and purity (>99%) evaluated by peptide sequencing and mass spectrometry (ESI-Ion trap LCQ DecaXP Plus by ThermoFinnigan). Peptides were lyophilized at −20° C. Concentration of the stock solution was estimated by amino acid analysis.

The molecular weights measured by mass spectrometry are listed in Table I below:

TABLE I

| SEQ ID N^{o}. | MW (g/mol) |
|---|---|
| 4 | 2 245.8 |
| 5 | 636.8 |
| 6 | 2865.5 |

### Example 2

### Biological Assays

In Vitro Assays of Activity.

The activity of compounds of the invention in inhibiting the formation of aggregated fibrils can be tested by following the changes in fluorescence signal of a fluorophore that has an affinity for the amyloid fibrils.

Amyloid formation can be quantitatively evaluated by the fluorescence emission of thioflavine T (ThT) bound to amyloid fibrils, as reported by Levine et al., 1993 and also Soto et al., 1995.

In this assay, peptides of the invention were solubilized in water at different concentration in small Eppendorff tubes and lyophilized.

Ab₁₋₄₂ (a synthetic peptide with the same sequence as the one deposited in the amyloid plaques in Alzheimer's brain, SEQ ID NO: 11) was solubilized at the concentration of 1 mg/ml in 2 mM NaOH. Aliquots were lyophilized (storage −80° C.). Several aliquots of Ab₁₋₄₂ at a concentration of 0.5 mg/ml (110 mM) were prepared in 0.1M Tris, pH 7.4 and incubated for 2 or 5 days at 37° C. in the absence or in the presence of different concentrations of the pre-lyophilized peptides of the invention (ranged from 10 mM to 1 mM). Thioflavin T was purchased from Sigma (T-3516). For example, 120 μg of Ab₁₋₄₂ and mixed to 1 μl of DMSO and 239 μl of 0.1M Tris, pH 7.4. From this solution, 120 μl are incubated 5 days at 37° C. and 120 μl are used to solubilize the peptide of the invention at the desired concentration and incubated 5 days at 37° C.

At the end of the incubation period, 50 mM Glycine, pH 9.2 and 2 μM ThT are added to the incubated mixture described above in a final volume of 2 ml (850 μl of pure water, 200 μl of 50 mM Glycine, pH 9.2 and 40 μl of Thioflavin T (1 mM in pure water) are added to 60 μl of sample.

Fluorescence is measured at excitation 435 nm and emission 485 nm in a Perkin Elmer, model LS50B fluorescence spectrometer. Measurements are carried out after the signal is stable for at least 1-2 min. The initial value of fluorescence represents the fluorescence obtained with Ab₁₋₄₂ peptide alone (highest concentration of fibrils) representing 100% of formed fibrils.

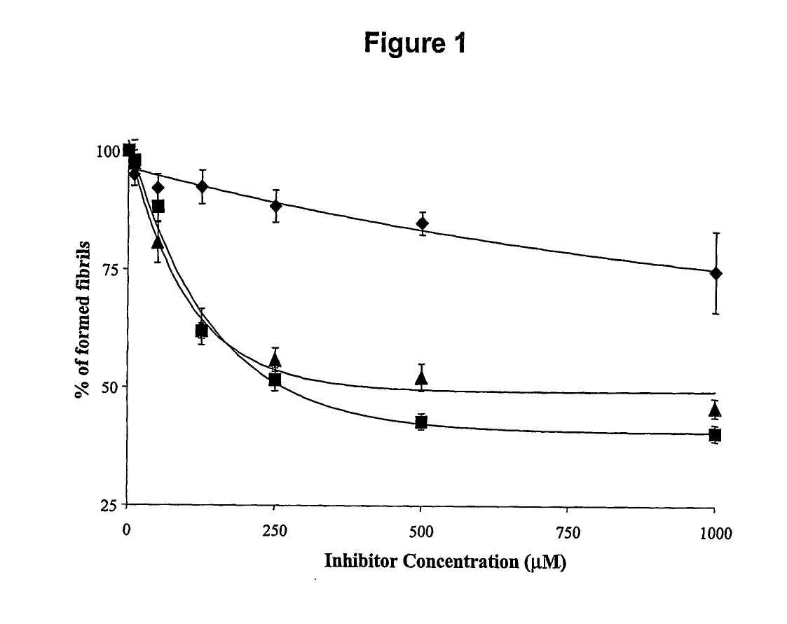

As shown on FIG. 1, peptides of the invention, pAntp (SEQ ID NO: 4) and pAntp-BSB1 (SEQ ID NO: 6), exhibit a high degree of inhibition of the fibrillogenesis process. Above 500 μM in peptide concentration, the % of fibrils in presence of peptides of the invention, pAntp peptide (SEQ ID NO: 4) and pAntp-BSB1 peptide (SEQ ID NO: 6), reaches a plateau of whereas in presence of BSB1 (SEQ ID NO: 5), the percentage of formed fibrils does not reach a plateau limit within these concentration ranges. In addition, the % of formed fibrils is much lower in presence of peptides of the invention.

The percentage of inhibition of Ab₁₋₄₂ fibril formation induced by compounds of the invention can be calculated using an analytical method such as described in Soto et al., 1998. Percentages of inhibition at a concentration of 500 μM in peptide of the invention are reported in Table II below:

TABLE II

| SEQ ID NO.: | % Inhibition of amyloid fibrils |
|---|---|
| 4 | 48 |
| 5 | 15 |
| 6 | 57 |

The inhibitory concentration at 50% of the effect (IC₅₀) of compound of the invention were calculated. The IC₅₀ values were then about 71 μM±28 and 98 μM±20 for pAntp (SEQ ID NO: 4) and for pAntp-BSB1 (SEQ ID NO: 6) respectively.

The data above indicate that peptides of the invention inhibit amyloid aggregates formation. In addition, a conjugate formed by peptide of the invention coupled covalently to a known beta-sheet breaker (BSB1) has a higher inhibiting effect on beta amyloid fibril formation than the beta-sheet breaker alone.

Cellular Assay of Activity.

Amyloid fibrils are cytotoxic, inducing cell death by apoptosis (Levine et al., 1993). The ability of the compounds of the invention in preventing the amyloid formation can be evaluated by measuring the decrease in the amyloid fibrils cytotoxicity in a cell assay. Toxicity was measured by comparing the effects of samples of Ab₁₋₄₂ (SEQ ID NO: 11) alone or of mixtures of Ab₁₋₄₂ combined with the peptides of the invention, on the reduction of the redox active dye, 3-(4,5-dimethylthiazol-2-yl)-2,5-diphenyltetrazolium bromide (MTT) by PC12 cells. PC-12 cells (ATCC) were grown in medium containing 85% of RPMI 1640, 5% fetal bovine serum, 10% heat-inactivated horse serum, 3.6 mM L-glutamine, in an humidified incubator at 37° C. and 5% CO₂.

Peptides of the invention were solubilized in water at different concentration in small Eppendorff tubes and lyophilized.

Ab₁₋₄₂ is solubilized at the concentration of 1 mg/ml in 2 mM NaOH. Aliquots are lyophilized (storage −80° C.). Aliquots of Aβ₁₋₄₂ (SEQ ID NO: 11) at a concentration of 0.5 mg/ml (110 μM) prepared in 0.1M Tris, pH 7.4 are incubated alone or in the presence of different concentrations of pre-lyophilized peptides of the invention (ranged from 0.030 μM to 10 μM) for 36 h at 37° C., gently swirled on a rotary shaker.

At the end of the incubation period, the medium of PC12 cells (10000-15000 cells/well) is slowly removed and replaced by an aliquot of the solution containing 5 μl of sample Aβ₁₋₄₂ alone or with peptide of the invention and 95 μl of medium to reach a final concentration of Aβ₁₋₄₂ of 5.5 μM in the well. The cells are incubated for 24 h and thereafter the cellular viability was evaluated using the MTT kit (Kit I (MTT), No. 1 465 007 Roche, Mannheim, Germany). Levels of reduced MTT are determined by measuring the difference in absorbance at 595 and 650 nm using a microplate reader and the extend of cellular viability is then deduced

Maximum fibril inhibition is obtained at a peptide concentration of 8 mM for the reference compound of SEQ ID NO: 5 in the fibrillogenesis assay described above. Therefore, the incubation preparation corresponding to this peptide concentration is diluted 20 times and added to the PC12 cells in order to measure the cellular viability in presence of such a mixture. The resulting cellular viability is set to a percentage of 100.

Cellular viability is then measured for peptides of the invention by adding the fibrillogenesis assay mixtures containing 1 mM of peptide of the invention (concentration where maximum fibril inhibition is obtained for peptides of the invention) to the PC12 cells after a 20-fold dilution.

Cellular viability in presence of peptides of the invention (SEQ ID NO: 4 and 6) is then expressed as a percentage of the cellular viability obtained in presence of the reference peptide of SEQ ID NO: 5 at a concentration corresponding to maximum fibrillogenesis inhibitory effect (set to 100%).

The corresponding percentage of cellular viability for the reference peptide of SEQ ID NO: 5 at this concentration is 4%.

Percentages of the cellular viability for peptides of the invention are presented in Table III below:

TABLE III

| SEQ ID N^{o}. | % cell viability |
|---|---|
| 4 | 132 |
| 6 | 166 |

The data above indicate that peptides of the invention increase cellular viability in presence of toxic amyloid fibril at very low peptide concentration.

Peptide of the invention (SEQ ID NO: 4) and conjugate thereof (SEQ ID NO: 6), formed by peptide of the invention coupled to a known beta-sheet breaker (pAntp-BSB1), have a higher inhibiting effect on the beta-amyloid cellular toxicity than the beta-sheet breaker itself (BSB1).

### REFERENCES

Bodanzsky 1993, Peptide Chemistry: A Practical Textbook 2^{nd} Revised Edition, Springer-Verlag Telos;
Cleland et al. 2001, Curr. Opin. Biotechnol., 12: 212-9;
Derossi et al. 1994, J. Biol. Chem., 269, 10444-10450;
Dougherty 2000, Curr. Opin. Chem. Biol., 4: 645-652;
Gennaro et al. 2000, of Remington's Pharmaceutical Sciences, Part 8, 20^{th} Edition, Marck Publishing Company, Easton, Pa.;
Golebiowski et al. 2001, Curr. Opin. Drug Discov. Devel., 4: 428-434;
Hardy et al. 2002, Science 297, 253-356;
Harper et al. 1997, Annu. Rev. Biochem., 66, 385-407;
Jordan, 2000, Semin. Neurol., 20, 179-85;
Karsa et al. 1993, (Ed), Encapsulation and Controlled Release; Stephenson (Editor), Springer Verlag;
Kim et al. 2000, Comb. Chem. High Throughput Screen, 3: 167-183;
Levine et al. 1993, Prot. Sci., 2, 404-410;
Levy et al. 1990, Science 248, 1124-1126;
Luo et al. 2001, Exp. Opin. Ther. Patents, 11: 1395-1410;
Lowe et al. 2001, Biochemistry, 40, 7882-7889;
Mann 1988, Histopathology 13, 125-37;
Maury 1995, Lab Invest. 72, 4-16;
Rousselle et al. 2000, Mol. Pharmacol. 57, 679-686;
Selkoe 1997, Science 275, 630-631;
Soto et al. 1995, J. Biol. Chem. 270, 3063-3067;
Soto et al. 1998, Nature Medicine, 4(7) 1998:822-6;
Soto 1999, J. Mol. Med. 77, 412-418;
Talaga 2001, Mini Review in Medicinal Chemistry, 1, 175-186;
Villain et al. 2001, Chem. Biol., 8: 673-9;
Xia et al. 2003, Current Opinion in Investigational Drugs, 4(1), 55-59;
Yacobi et al. 1998, Oral Sustained Release Formulations: Design and Evaluation, Eva Halperin-Walega (Editor), 1st Ed. edition; Pergamon Press;
Yankner 1996, Neuron 16, 921-932;
Weng et al. 2000 (Editor), FMOC Solid Phase Peptide Synthesis: A Practical Approach Oxford University Press;
WO 96/39834, New York University;
WO 98/38861 The Trustees of Colombia University in The City of New York;
WO 00/29427 Cyclacel Ltd;
WO 00/63246 Adherex Technologies Inc.;
WO 01/07473 Stott Kelvin;
WO 01/09170 CNRS;
WO 01/34631 Axonyx Inc.;
WO 02/062989 Sequitur Inc.
WO 02/10195 Theratechnologies Inc.;
FR 2829940 Synt:em;
U.S. Pat. No. 6,319,498 Praecis Pharmaceuticals Inc.

1. A peptide or a salt thereof comprising an amino acid sequence of Formula I (SEQ ID NO: 1):
X₁[Lys X₂ X₃ Phe Gln]ₘArg Gln Ile[Lys X₄ Pro Phe Gln]ₙX wherein
X₁ is absent or is an acetyl group;
X₂ and X₄ are independently selected from the group consisting of Ileu and Leu;
X₃ is selected from the group consisting of Pro and Trp;
X is a peptidic moiety of a length selected from the group consisting of 1, 2, 3, 4, 5, 6, 7 and 8 amino acids wherein X comprises at least one basic amino acid and wherein X is amidated at the C-terminus;
m is an integer selected from the group consisting of 0 and 1;
n is an integer selected from the group consisting of 1 and 2;.

2. The peptide of claim 1, wherein X is a peptidic moiety of a length selected from the group consisting of 5, 6, 7 and 8 amino acids and wherein X comprises at least one basic amino acid.

3. The peptide according to claim 1, wherein X comprises at least one basic amino acid selected from the group consisting of Lys and Arg.

4. The peptide according to claim 1, wherein X is the peptidic moiety (SEQ ID NO: 2):
Asn X₅ X₆ Met X₇ Trp X₈ X₉—NH₂ wherein
X₅, X₆, X₇, X₈ and X₉ are independently selected from the group consisting of Arg and Lys.

5. The peptide according to claim 1 wherein X is SEQ ID NO: 10.

6. The peptide according to claim 1, wherein m is 0 and n is 1.

7. The peptide according to claim 1 wherein X₁ is acetyl.

8. The peptide according to claim 1, wherein m is 0 and n is 2.

9. The peptide according to claim 1, wherein m is 1 and n is 1.

10. The peptide according to claim 1 selected from the group consisting of SEQ ID NO: 7 and SEQ ID NO: 8.

11. (canceled)

12. A pharmaceutical composition comprising the peptide of claim 1 and a pharmaceutically acceptable excipient, diluent, carrier, or combination thereof.

13. A method of treating at least one of Alzheimer's disease, Dementia pugilistica, Hereditary Cerebral Haemorrage with amyloidosis of the Dutch type (HCHWA-D), head trauma, and vascular dementia with amyloid angiopathy, in a subject in need thereof, comprising
administering a peptide, or a salt thereof, of Formula (II) (SEQ ID NO: 3):
X₁ [Lys X₂ X₃ Phe Gln]ₘ Arg Gln Ile [Lys X₄ X₅ Phe Gln]ₙ X to the subject in need thereof in an amount sufficient to treat one of Alzheimer's disease. Dementia pugilistica, Hereditary Cerebral Haemorrage with amyloidosis of the Dutch type (HCHWA-D), head trauma, and vascular dementia with amyloid angiopathy, wherein
X₁ is absent or is an acetyl group;
X₂ and X₄ are independently selected from the group consisting of Ile and Leu;
X₃ and X₅ are independently selected from is the group consisting of Pro and Trp;
X is a peptidic moiety of a length selected from the group consisting of 1, 2, 3, 4, 5, 6, 7 and 8 amino acids wherein X comprises at least one basic amino acid and wherein X is amidated at the C-terminus;
m is an integer selected from the group consisting of 0 and 1;
n is an integer selected from the group consisting of 1 and 2.

14. The method of claim 13 wherein X₅ is Trp.

15. The method of claim 13 wherein the peptide of Formula (II) is of SEQ ID: 1.

16. The method of claim 13 wherein X₄ is Ile.

17. The method of claim 13 wherein X is a peptidic moiety of a length selected from the group consisting of 5, 6, 7 and 8 amino acids and wherein X comprises at least one basic amino acid.

18. The method of claim 13 wherein X comprises at least one basic amino acid selected from the group consisting of Lys and Arg.

19. The method of claim 13 wherein X is (SEQ ID NO: 2).

20. The method of claim 13 wherein X is SEQ ID NO: 10.

21. The method of claim 13, wherein m is 0 and n is 1.

22. The method of claim 13 wherein X₅ is Trp, X is SEQ ID NO: 2, m is 0 and n is 1.

23. The method of claim 13, wherein m is 1 and n is 1.

24. The method of claim 13 wherein X is of SEQ ID NO: 4.

25. The method of claim 13 wherein X is selected from the group consisting of SEQ ID NO: 7, SEQ ID NO: 8 and SEQ ID NO: 9.

26. The method of claim 13, wherein the disease is Alzheimer's disease.


SEQUENCE LISTING (13 sequences)

SEQ ID NO: 1 - protein, 26 aa, Artificial Sequence
  Description of Artificial Sequence Synthetic peptide
  MOD_RES (1): May or may not be present
  MOD_RES (2): Ile, Leu or not present
  MOD_RES (3): Pro, Trp or not present
  MOD_RES (4)..(5): May or may not be present
  MOD_RES (10): Ile or Leu
  MOD_RES (14): May or may not be present
  MOD_RES (15): Ile, Leu or not present
  MOD_RES (16)..(18): May or may not be present
  MOD_RES (19)..(26): Variable amino acid and this region may encompass 1 to 8 residues
  N-term may or may not be acetylated
  C-term amidated
  see specification as filed for detailed description of substitutions and preferred embodiments
KXXFQRQIKXPFQKXPFQXXXXXXXX

SEQ ID NO: 2 - protein, 8 aa, Artificial Sequence
  Description of Artificial Sequence Synthetic peptide
  MOD_RES (2)..(3): Arg or Lys
  MOD_RES (5): Arg or Lys
  MOD_RES (7)..(8): Arg or Lys
  C-term amidated
NXXMXWXX

SEQ ID NO: 3 - protein, 26 aa, Artificial Sequence
  Description of Artificial Sequence Synthetic peptide
  MOD_RES (1): May or may not be present
  MOD_RES (2): Ile, Leu or not present
  MOD_RES (3): Pro, Trp or not present
  MOD_RES (4)..(5): May or may not be present
  MOD_RES (10): Ile or Leu
  MOD_RES (11): Pro or Trp
  MOD_RES (14): May or may not be present
  MOD_RES (15): Ile, Leu or not present
  MOD_RES (16): Pro, Trp or not present
  MOD_RES (17)..(18): May or may not be present
  MOD_RES (19)..(26): Variable amino acid and this region may encompass 1 to 8 residues
  N-term may or may not be acetylated
  C-term amidated
  see specification as filed for detailed description of substitutions and preferred embodiments
KXXFQRQIKXXFQKXXFQXXXXXXXX

SEQ ID NO: 4 - protein, 16 aa, Artificial Sequence
  Description of Artificial Sequence Synthetic peptide
  C-term amidated
RQIKIWFQNRRMKWKK

SEQ ID NO: 5 - protein, 5 aa, Artificial Sequence
  Description of Artificial Sequence Synthetic peptide
  N-term acetylated
  C-term amidated
LPFFD

SEQ ID NO: 6 - protein, 21 aa, Artificial Sequence
  Description of Artificial Sequence Synthetic peptide
  C-term amidated
RQIKIWFQNRRMKWKKLPFFD

SEQ ID NO: 7 - protein, 16 aa, Artificial Sequence
  Description of Artificial Sequence Synthetic peptide
  N-term acetylated
  C-term amidated
RQIKIPFQNRRMKWKK

SEQ ID NO: 8 - protein, 21 aa, Artificial Sequence
  Description of Artificial Sequence Synthetic peptide
  N-term acetylated
  C-term amidated
RQIKIPFQKIPFQNRRMKWKK

SEQ ID NO: 9 - protein, 21 aa, Artificial Sequence
  Description of Artificial Sequence Synthetic peptide
  N-term acetylated
  C-term amidated
KIWFQRQIKIWFQNRRMKWKK

SEQ ID NO: 10 - protein, 8 aa, Artificial Sequence
  Description of Artificial Sequence Synthetic peptide
  C-term amidated
NRRMKWKK

SEQ ID NO: 11 - protein, 42 aa, Homo sapiens [sequence omitted]

SEQ ID NO: 12 - protein, 4 aa, Artificial Sequence
  Description of Artificial Sequence Synthetic peptide
KKKK

SEQ ID NO: 13 - protein, 4 aa, Artificial Sequence
  Description of Artificial Sequence Synthetic peptide
EEEE